(19) United States
(12) Patent Application Publication
(10) Pub. No.: US 2019/0000375 A1
(43) Pub. Date: Jan. 3, 2019

(54) METHOD TO INCREASE AHI ESTIMATION ACCURACY IN HOME SLEEP TESTS

(71) Applicant: KONINKLIJKE PHILIPS N.V., Eindhoven (NL)

(72) Inventors: Pedro Miguel FERREIRA DOS SANTOS DA FONSECA, Borgerhout (BE); Xavier Louis Marie Antoine AUBERT, Brussels (BE)

(21) Appl. No.: 16/020,309

(22) Filed: Jun. 27, 2018

Related U.S. Application Data
(60) Provisional application No. 62/526,748, filed on Jun. 29, 2017.

(51) Int. Cl. A61B 5/00; A61B 5/0205; A61B 5/024

(52) U.S. Cl. CPC A61B 5/4818; A61B 5/0205; A61B 5/4815; A61B 5/4812; A61B 5/02416

(57) ABSTRACT

A method of determining sleep statistics for a subject includes the steps of: collecting cardio-respiratory information of the subject; extracting features from the cardio-respiratory information; determining sleep stages of the subject by using at least some of the extracted features; determining an estimated total sleep time of the subject based on the determined sleep stages; and determining sleep statistics of the subject using the estimated total sleep time.

### CROSS-REFERENCE TO PRIOR APPLICATIONS

This application claims the benefit of U.S. Provisional Application No. 62/526,748, filed on 29 Jun. 2017. This application is hereby incorporated by reference herein.

### BACKGROUND OF THE INVENTION

### 1. Field of the Invention

The present invention pertains to methods for determining sleep statistics for a patient, and more particularly to a method to increase AHI estimation accuracy in home sleep tests which utilizes an improved method of determining a patient's total sleep time.

### 2. Description of the Related Art

Home sleep tests (HSTs) rely on unobtrusive techniques for recording vital signals and other physiological measurements so that the subject can be monitored at home without perturbing daily habits and comfort. An important parameter of such sleep tests is the total time in which the subject is actually sleeping, which is typically referred to as the total sleep time. Examples of sleep statistics requiring the total sleep time are given by the Apnea-Hypopnea index (AHI), which is a key parameter for sleep disordered breathing diagnosis, or the sleep efficiency parameter providing a first objective measure of sleep quality or the Periodic-Limb-Movement index (PLMI). Another example is given by the arousal index defined as the mean number of cortical arousals per hour of sleep. In all these cases, specific events have to be detected and counted, for example, in terms of obstructive sleep apneas (OSA) or significant limb movements or arousals, and the mean number of such events per hour of sleep is obtained by normalizing with the total sleep time, after discarding the wake intervals. Specific methods for detecting these target events will not be considered here, as these are “state-of the-art” and can be reliably obtained from usual sensors such as an SpO2 finger-clip or an impedance thorax-belt or an accelerometer placed on the ankle.

Presently, sleep/wake classification may be attempted by simple actigraphy techniques, based on the absence of movement characterizing sleep. However, this is just a necessary condition and not a sufficient one, since subjects affected by insomnia might well stay still while not sleeping. Hence, actigraphy is known for over-estimating the true sleep time of problem sleepers, which in turns lead to an underestimation of sleep-statistics requiring an average number of events per hour of sleep. An improved sleep/wake classification requires identifying the underlying sleep stages (REM, non-REM, wake, etc.) so that true sleep states can be reliably discriminated versus non-sleep states. In this context, the total sleep time is actually a by-product of the whole sleep-stage analysis, which can be used for other purposes like deriving objective measures of sleep quality, or providing refined sleep diagnosis related to a reduction or even an absence of REM or deep sleep, well beyond the sole AHI or PLMI parameter values.

Given the high prevalence of sleep breathing disorders (SDB) in the general population, it is important to remind a number of elements that are truly part of this invention background. Sleep breathing disorders are caused by short repeated events like obstructive or central apneas and hypopneas, leading to a temporary reduction or cessation of the respiration process. Such events may remain unnoticed by the subject as long as sleep-efficiency is not strongly reduced. This explains why sleep respiration disorders remain under-diagnosed and are often only identified at a later severe stage when the subject is really sleep deprived to the extent that normal life (including professional activity) is dramatically impaired. The key parameter for SDB diagnosis is the Apnea-Hypopnea Index (AHI) defined as the ratio of the number of detected apnea/hypopnea respiratory events divided by the total sleep time. Automatic detection of apnea and hypopnea events is typically based on a dual signal input from the respiration effort and SpO2 finger clip, such as described in “Home Diagnosis of Sleep Apnea: A Systematic Review of the Literature,” Chest, vol. 124, no. 4, pp. 1543-79, 2003, the contents of which are incorporated herein by reference. The first signal leads to the amplitude variations of respiratory movements while the second measurement provides relative oxygen desaturation levels. This enables detection of temporary reduction or cessation of respiration movements and at the same time to quantification of the impact of these events on blood oxygenation.

Obstructive sleep apnea (OSA) has been associated with an increased risk of cardiac and cerebrovascular diseases such as hypertension, heart failure, arrhythmias, myocardial ischemia and infarction, pulmonary arterial hypertension and renal disease, metabolic dysregulation (insulin resistance and lipid disorders) and changes in cerebral blood flow and cerebral auto-regulation, which in turn are risk factors for cardiovascular diseases, stroke, dementia and cognitive impairment in the elderly. OSA patients with daytime sleepiness have also been found to be more prone to motor- and work-accidents and are be less productive at work. Early studies estimated the prevalence at 2% for women, and 4% for men, however, more recent reviews claim that roughly 1 of every 5 adults has at least mild OSA and 1 of every 15 has at least moderate OSA. In the context of frequent overweight and obesity cases, prevalence of SDB is likely to increase further.

However, studies have found that more than 85% of patients with clinically significant OSA remain undiagnosed. The reason for this level of under-diagnosis is multi-factorial, although one possible explanation may lie on the difficulty to accurately screen for the presence and severity of OSA. Although diagnosis is typically established by means of full-night polysomography (PSG) studies, such studies are complex and very expensive procedures that often represent a high burden for the patient. Not only do such studies remove the patients from their typical sleep environment, but such studies are also known for severely disrupting sleep, possibly giving an unrepresentative view of a possible disorder.

Recent years have seen the increase in popularity of home sleep tests (HSTs). HSTs typically comprise a smaller set of sensors than a PSG, typically an ‘SpO2’ sensor, a respiratory effort belt, and respiratory flow sensor on nose/mouth. This makes such tests more comfortable and easier to set up. Furthermore, due to their portability, they can be used at home, where they are installed by the subject before going to bed, and removed after they wake up in the morning. After the devices are returned to the referring physician, the data is often manually or (semi-) automatically analyzed, and amongst others, parameters such as the Apnea-Hypopnea Index (AHI, average number of apnea/hypopnea events per hour of sleep) and Sleep efficiency (SE-%, percent of true sleep time per hour of time in bed) are calculated, from which the treating physician can make a first diagnosis.

Sleep stages are traditionally annotated, manually or (semi-)automatically from EEG signals recorded during PSG in a sleep laboratory, which is expensive and labor intensive. However, it has been recently shown that cardiorespiratory information provides a promising alternative to EEG, with the benefit that it can be measured unobtrusively. Cardiorespiratory-based sleep stage classification has been increasingly studied over the past years. Many studies have reported results on the classification of different sleep stages using these types of features. Such methods typically make use of heart rate variability features derived from a cardiac signal, augmented with respiratory information from a thorax belt or nasal flow sensor, and body movements, typically measured from an accelerometer or an actigraphy device. Although HSTs do not have all the information otherwise available in a traditional PSG, e.g. in a sleep clinic, they have the potential of reducing the gap between full PSG and simple actigraphy, while offering an increased comfort at a reduced cost. With the recent HST devices now equipped with most common sensors, a large part of the PSG-derived sleep-staging information becomes available. More precisely, HST-based sleep-staging methods leads to improved estimations of sleep and wake times and offer a much better alternative for the computations of AHI or PLMI values.

Indeed, important diagnostic parameters that depend on averaging over the entire night (such as AHI or PLMI), are currently, with most available HST devices, normalized based on the total recording time, instead of the total sleep time, which for subjects with low sleep efficiency (low number of sleep hours versus total time spent in bed), leads to severe underestimations of these values and consequently, under-diagnosis of the severity or even the presence of (sleep breathing) disorders. Accordingly, a need exists for systems and methods which can provide improved measurements of a subjects total sleep time.

### SUMMARY OF THE INVENTION

Embodiments of the present invention provide for improved estimations of the total sleep time of a subject based on a sleep-stage analysis to identify true sleep intervals versus wake intervals. Accordingly, it is an object of the present invention to provide a method of determining sleep statistics for a subject. The method comprises: collecting cardio-respiratory information of the subject; extracting features from the cardio-respiratory information; determining sleep stages of the subject by using at least some of the extracted features; determining an estimated total sleep time of the subject based on the determined sleep stages; and determining sleep statistics of the subject using the estimated total sleep time.

Determining an estimated total sleep time of the subject based on the determined sleep stages may comprise: determining a duration of each sleep stage; and summing the durations of the sleep stages.

Collecting cardio-respiratory information of the subject may comprise collecting cardio-respiratory information via a home sleep testing device.

Extracting features from the cardio-respiratory information may comprise extracting at least one of: heart rate variability features, respiratory variability features, or body movements.

Collecting cardio-respiratory information of the subject may comprise collecting heart rate information using a SpO2 sensor.

Collecting cardio-respiratory information of the subject may comprise collecting respiratory effort using a thoracic belt.

Collecting cardio-respiratory information of the subject may comprise collecting respiratory effort using a thoracic belt and a SpO2 sensor.

The method may further comprise collecting information regarding body movement of the subject via an accelerometer.

The method may further comprise determining information regarding body movement via information received from one or more of a respiratory thoracic belt and a SpO2 sensor.

The method may further comprise providing an indication of one of more of the determined sleep stages to the subject.

It is another object of the present invention to provide a machine readable medium encoded with a computer program comprising program code for implementing the methods described herein.

It is yet another object of the present invention to provide a computer program product including a non-transitory machine readable medium encoded with a computer program comprising program code for implementing the methods described herein.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a block diagram showing implementation of an example embodiment of the present invention; and

FIG. 2 is a flow chart showing the general steps of a method in accordance with an example embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the term “feature” is used to describe a physiological characteristic of relevance, computed with statistical or signal processing techniques from the raw measurements collected by the considered sensor(s). For example, cardiac activity can be measured with sensors providing a single-lead ECG, and, after a number of signal processing and statistical analysis steps meant for detecting the location and timing of individual heart beats, a “feature” describing the “average heart rate” of a person over a specified time period can be obtained. This feature is usable in a classifier such as the one described in this invention for the purpose of sleep analysis, whereas the raw signal ECG is not.

As used herein, the term “epoch” shall mean a standard 30 second duration of a sleep recording that is assigned a sleep stage designation. The choice of an epoch length of 30 seconds was done to match the 30 second epochs recommended by the American Academy for Sleep Medicine (AASM), for sleep scoring. By extracting features on a basis of non-overlapping 30 seconds segments, sleep stages can be classified with the same time resolution, and match the criteria recommended by AASM. It is to be appreciated, however, that epochs of other duration may be employed without varying from the scope of the present invention.

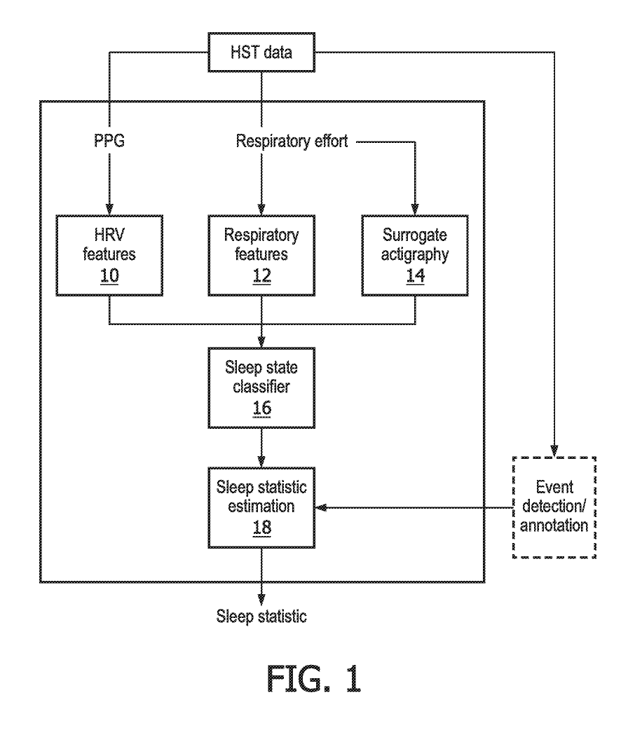

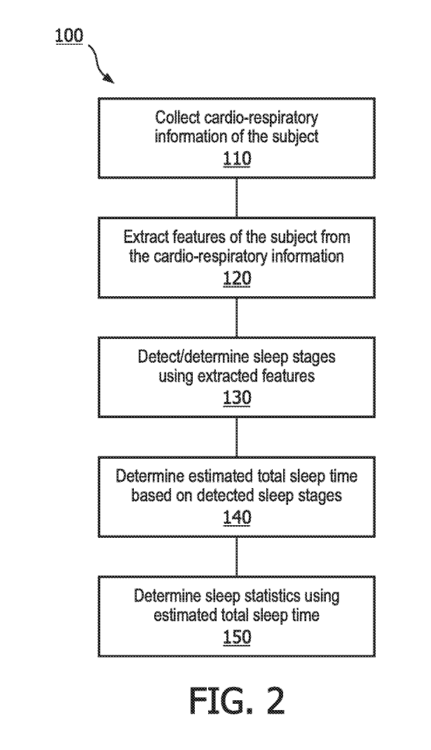

FIG. 1 illustrates a block diagram describing implementation of an example embodiment of the present invention. Common HST devices such as, for example, without limitation, the Philips Alice NightOne device, have a finger-mounted SpO2 sensor which can measure photoplethysmography (PPG), a respiratory effort sensor (respiratory inductance plethysmography (RIP) belt) and respiratory flow (nose/mouth thermistor). FIG. 2 illustrates a flow chart showing the general steps of a method 100 in accordance with an example embodiment of the present invention. In this example embodiment, as shown in step 110, cardio-respiratory information of the subject (patient) are collected (such as via an HST device). Next, as shown at step 120, a plurality of features of the subject of the home sleep test are extracted (examples of which are described herein below) which describe characteristics of: heart rate variability, respiratory variability and body movements. Heart rate variability features (i.e., HRV features 10) are measured from heart beats detected from the raw PPG signal recorded with the SpO2 sensor. Respiratory variability features (i.e., Respiratory features 12) are measured from the respiratory effort signal recorded with the thoracic belt. In the absence of recorded accelerometer signals, body movements may be derived from artifacts in the respiratory effort signal in order to obtain surrogate actigraphy features 14 using techniques such as described in WO2016/07182 A1 to Fonseca, the contents of which are incorporated herein by reference. If the invention is embodied in an HST which can record accelerometer or actigraphy signals, these can be used instead of computing the surrogate actigraphy 14, currently measured from the respiratory effort signal.

Following step 120, a number of the features extracted in step 120 are input to a sleep state classifier 16 in order to detect/classify sleep stages of the subject, as shown in step 130. The sleep state classifier is trained in advance using data collected from a variety of subjects with different characteristics, ranging from healthy to disordered breathing subjects, with mild, moderate and severe sleep apnea. The training procedure exploits ground-truth data, manually annotated by one or more human specialists according to the recommendations of the American Academy of Sleep Medicine (AASM), using any machine learning technique fed with the extracted “features”, as described in the literature. The pre-computed models, based on this ground-truth exemplary data, are then used to perform the automatic classification of new, “never seen before” data collected with the device during its actual usage. The machine learning techniques, used to train models applied later in this invention, associate patterns from the cardiorespiratory features to examples of human-annotated sleep stages observed in the pre-processed training data.

The training set is crucial for a successful use of this invention, accordingly the training set should comprise a balanced number of example recordings from each group. After the sleep states are detected and classified for a complete recording, the estimated total sleep time can be determined, as shown in step 140, by summing the times of each of the sleep stages detected in step 130. The total sleep time, along with sleep events detected/determined from the collection of step 110, is then used by a sleep statistic estimator to provide sleep statistics, such as shown in step 150. The sleep statistic estimator 18 takes as input sleep events (for example the number of apneas and hypopneas) manually or (semi) automatically annotated and calculates statistics regarding the estimated sleep time obtained by summing the total time with detected sleep state. In this example, this results in the average number of events per hour of sleep (for example, without limitation, the average number of apnea or hypopnea events per hour of sleep-apnea-hypopnea index, or AHI). This example can of course be used for other statistics, such as the arousal rate (average number of arousals per hour of sleep), period limb movement index (average number of periodic limb movements per hour of sleep), etc.

It is to be appreciated that algorithmic components described herein are typically integrated in a software program and executed by a computer processor or other suitable processing device running on any suitable electronic device (e.g. personal computer, workstation), or dedicated medical device (e.g. including a processor that can directly perform the required calculations) or on a cloud service connected via Internet to any device with an interface for reporting the results.

The following describes examples of features which have been shown, in literature, to allow sleep stages to be automatically classified from recordings of cardiac, respiratory and body movement signals. The sleep state classifier 16 described herein uses a combination of one or more of these features in identifying sleep states, as determined during a training procedure.

Considering cardiac activity, we give examples of 92 cardiac features which can be computed from the beats detected from the PPG signal, more specifically from the time series comprised of consecutive heart beats, also referred to as inter-beat intervals (IBI). These include time domain features, for example computed over nine consecutive non-overlapping 30-second epochs, such as mean heart rate, detrended and non-detrended mean heartbeat interval, standard deviation (SD) of heartbeat intervals, difference between maximal and minimal heartbeat intervals, root mean square and SD of successive heartbeat interval differences, and percentage of successive heartbeat intervals differing by >50 ms, mean absolute difference and different percentiles (at 10%, 25%, 50%, 75%, and 90%) of detrended and non-detrended heart rates and heartbeat intervals as well as the mean, median, minimal, and maximal likelihood ratios of heart rates. Cardiac features also include frequency domain features such as the logarithmic spectral powers in the very low frequency band (VLF) from 0.003 to 0.04 Hz, in the low frequency band (LF) from 0.04 to 0.15 Hz, in the high frequency band (HF) between 0.15 to 0.4 Hz, and the LF-to-HF ratio, where the power spectral densities were estimated for example over nine epochs. The spectral boundaries can also be adapted to the corresponding peak frequency, yielding their boundary-adapted versions. They also include the maximum module and phase of HF pole and the maximal power in the HF band and its associated frequency representing respiratory rate. In addition, they include features describing non-linear properties of heartbeat intervals were quantified with detrended fluctuation analysis (DFA) over 11 epochs and its short-term, longterm, and all time scaling exponents, progressive DFA with non-overlapping segments of 64 heartbeats, windowed DFA over 11 epochs, and multi-scale sample entropy over 17 epochs (length of 1 and 2 samples with scales of 1-10).

Cardiac features also include approximate entropy of the symbolic binary sequence that encodes the increase or decrease in successive heartbeat intervals over nine epochs. In addition, they include features based on a visibility graph (VG) and a difference VG (DVG) method to characterize HRV time series in a two-dimensional complex network where samples are connected as nodes in terms of certain criteria. The network-based features can be computed over seven epochs, and comprise mean, SD, and slope of node degrees and number of nodes in VG- and DVG-based networks with a small degree (≤3 for VG and ≤2 for DVG) and a large degree (≥10 for VG and ≥8 for DVG), and assortativity coefficient in the VG-based network.

Finally, cardiac features can include Teager Energy, a method to quantify instantaneous changes in both amplitude and frequency, to detect and quantify transition points in the IBI time series. All of the aforementioned features were previously described in the context of cardiac or cardiorespiratory sleep staging and are either described in detail or referred to in the scholarly articles “Sleep stage classification with ECG and respiratory effort,” TOP Physiol. Meas., vol. 36, pp. 2027-40, 2015 or “Cardiorespiratory Sleep Stage Detection Using Conditional Random Fields,” IEEE J. Biomed. Heal. Informatics, 2016, the contents of which are both incorporated herein by reference.

Concerning respiratory activity, we give examples of 44 features which can be derived from respiratory effort, for example measured with (thoracic) RIP belt sensors. In the time domain, these features comprise the variance of respiratory signal, the respiratory frequency and its SD over 150, 210, and 270 seconds, the mean and SD of breath-by-breath correlation, and the SD in breath length. They also include respiratory amplitude features, including the standardized mean, standardized median, and sample entropy of respiratory peaks and troughs (indicating inhalation and exhalation breathing depth, respectively), median peak-to-trough difference, median volume and flow rate for complete breath cycle, inhalation, and exhalation, and inhalation-to-exhalation flow rate ratio. Besides, they include the similarity between the peaks and troughs by means of the envelope morphology using a dynamic time warping (DTW) metric. They also include respiratory frequency features, such as the respiratory frequency and its power, the logarithm of the spectral power in VLF (0.01-0.05 Hz), LF (0.05-0.15 Hz), and HF (0.15-0.5 Hz) bands, and the LF-to-HF ratio. They include respiratory regularity measures, obtained for example by means of sample entropy over seven 30-second epochs and self-(dis)similarity based on DTW and dynamic frequency warping (DFW) and uniform scaling. The same network analysis features as for cardiac features previously described can also be computed for breath-to-breath intervals.

Numerous studies have shown that the interaction between cardiac and respiratory activity varies across sleep stages. These features may be calculated simultaneously from IBI time series derived from PPG signals, or from respiratory effort signals, for example measured from RIP signals. These include for example the power associated with respiratory-modulated heartbeat intervals, quantified for example over windows of nine epochs, VG and DVG-based features for cardiorespiratory interaction and phase coordination between IBI and the respiratory period for different ratios.

The conventional way to measure body movements is to record them with an accelerometer, often integrated in a so-called actigraphy device. However, some HST devices such as, for example, without limitation, the Philips NightOne do not record body movements (although they often contain an accelerometer, used to detect lying position). In this case, we quantify the amount of body-movement induced artifacts present in other, measured, modalities as described in WO2016/07182 A1 and “Estimating actigraphy from motion artifacts in ECG and respiratory effort signals,” Physiol. Meas., vol. 37, no. 1, pp. 67-82, 2016. Such approach allows the quantification of gross body movements with similar meaning as those measured by an actigraphy device to be used instead.

In order to use one or more of the previously described features to automatically classify sleep stages, traditional machine learning algorithms can be used. These can include Bayesian linear discriminants, such as described (for example without limitation) in “Sleep stage classification with ECG and respiratory effort,” IOP Physiol. Meas., vol. 36, pp. 2027-40, 2015 and “Cardiorespiratory Sleep Stage Detection Using Conditional Random Fields,” IEEE J. Biomed. Heal. Informatics, 2016, or more advanced probabilistic classifiers such as (for example, without limitation) those described in WO2016/097945 (the contents of which are incorporated herein by reference) and “Cardiorespiratory Sleep Stage Detection Using Conditional Random Fields,” IEEE J. Biomed. Heal. Informatics, 2016. In practice, any classifier which, based on a pre-trained model and a set of features in a time series, can either classify two classes (to distinguish sleep and wake), or multiple classes (to distinguish further sleep stages, such as wake, N1 sleep, N2 sleep, N3 sleep and REM, or any simplifications such as wake, light sleep—N1 and N2 combined, N3 sleep and REM, or even wake, non-REM, and REM) can be used in this invention.

An example embodiment of the present invention will now be used to illustrate the potential of sleep-stage classification in a sleep-disordered population, and the improvements it gives in the estimation of disorder-related statistics. Training a Bayesian linear discriminant classifier on a training set comprising 414 recordings of healthy subjects and subjects suffering from different severities of obstructive sleep apnea, and then using the trained classifier on a hold-out set comprising 96 recordings (including PSG and reference annotations) of subjects with different severities of obstructive sleep apnea, the sleep stage classification performance indicated in

Table 1 and Table 2 below for a 4- and 3-class sleep stage classification problem, respectively, were obtained. To evaluate the performance against reference sleep stage annotations, traditional metrics of accuracy (percentage of correctly classified epochs) and Cohen's kappa coefficient of agreement, which gives an estimate of classification performance, compensated for change of random agreement, were used.

TABLE 1
Sleep stage classification performance for 4 classes
(wake, N1-N2 combined, N3 and REM sleep)

| N | Kappa (—) | Accuracy (%) |
|---|---|---|
| 96 | 0.50 ± 0.13 | 66.8 ± 8.6 |

TABLE 2
Sleep stage classification performance for 3 classes
(wake, non-REM and REM sleep)

| N | Kappa (—) | Accuracy (%) |
|---|---|---|
| 96 | 0.59 ± 0.13 | 78.6 ± 7.5 |

Regarding the estimation of sleep statistics, the AHI was computed based on reference annotations of the number of apneas and hypopneas on each recording, from which the average number of events per total recording time we calculated and, using the estimations of sleep time based on the classification results, the average number of events per total sleep time. The two estimations were then compared against a reference AHI obtained, for the same recordings, from the reference PSG data. The performance was compared with reference AHI using two conventional metrics: root-mean-squared error (RMS) and bias (average error). In addition, traditional clinical thresholds were used for the diagnosis of presence and severity of sleep disordered breathing, to evaluate the agreement with the reference diagnosis (established based on PSG). Using the thresholds AHI<5: no disorder, 5≤AHI<15: mild, 15≤AHI<30: moderate, AHI≥30: severe, the Cohen's kappa coefficient of agreement and the accuracy between the severity class established with the AHI estimated with total recording time and total sleep time and the reference AHI annotated based on PSG were calculated. All results are indicated in Table 3 below.

TABLE 3
AHI estimation error

| | | | Severity | Severity |
|---|---|---|---|---|
| | | | agreement: | agreement: |
| | RMS | Bias | kappa (—) | accuracy (%) |
| AHI error, estimated | 7.30 | −4.41 | 0.76 | 82.3% |
| with total recording time | | | | |
| AHI error, estimated | 4.05 | −0.93 | 0.85 | 88.5% |
| with total sleep time | | | | |

It is to be appreciated from Table 3 that there is a substantial decrease in the RMS error in AHI estimation, and an important decrease in the negative bias. While the AHI estimated with total recording time had a consistent underestimation of AHI of −4.41, using the estimation of AHI based on total sleep time the bias decreases to −0.93. To emphasize the importance of this improvement, it should be noted that an AHI of 5 is often used as a threshold to clinically decide upon the presence or absence of sleep apnea. An underestimation of 4.4 is critically close to this threshold, and may lead to under-diagnosis in case of subjects with low sleep efficiency where the difference between total recording and total sleep time is large.

As alternative or optional embodiments, it should be mentioned that the respiratory features can be calculated using the signals of different sensors. Although the example embodiment provided estimates respiratory features from RIP signals, these can also be calculated from signals such as respiratory flow (also typically part of the sensor set up of HST devices), or even surrogate measures of respiratory effort which can be obtained from sensors such as PPG, or ECG, such as described in “Respiration Signals from Photoplethysmography,” Anesth. Analg., vol. 117, no. 4, pp. 859-65, 2013 and “Clinical validation of the ECG-derived respiration (EDR) technique,” Comput. Cardiol., vol. 13, pp. 507-510, 1986, the contents of which are incorporated herein by reference.

Additionally, it should be emphasized that the cardiac features can also be calculated with signals from different sensors, such as ECG, or ballistocardiographic (BCG) sensors typically installed on or under the bed mattress. In these cases, the heart beat interval time series used to calculate the cardiac features are computed based on detected QRS complexes (in the case of ECG), or heart beats (in the case of BCG).

As optional embodiments, the current invention could also be used to compute sleep statistics during specific sleep stages (e.g. non-REM versus during REM sleep). These metrics, typically available only with a complete PSG, can aid the diagnosis of different sleep-stage specific disorders.

As another optional embodiment, if the HST comprises an accelerometer with which the lying/sleeping position can be detected, the current invention can also be used to improve the estimation of body position-dependent statistics. Here the advantage is, once more, that the accuracy of these statistics can be improved by basing them on total sleep time instead of total recording time.

It is to be appreciated that embodiments of the present invention are readily applicable to HST devices such as the Philips NightOne HST device, but also to any other sleep monitoring device which has the capability of measuring cardiac and/or respiratory activity and body movements and which is intended to estimate sleep statistics which can be relevant for the diagnosis or assessment of sleep disorders.

It is to be appreciated that the operations and methods described herein may be readily encoded, in whole or in-part, on machine readable storage medium(s) which may be readily employed by a processing device or devices to automatically carry out all or portions of the methods described herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of determining sleep statistics for a subject, the method comprising:
collecting cardio-respiratory information of the subject;
extracting features from the cardio-respiratory information;
determining sleep stages of the subject by using at least some of the extracted features;
determining an estimated total sleep time of the subject based on the determined sleep stages; and
determining sleep statistics of the subject using the estimated total sleep time.

2. The method of claim 1, wherein determining an estimated total sleep time of the subject based on the determined sleep stages comprises:
determining a duration of each sleep stage; and
summing the durations of the sleep stages.

3. The method of claim 1, wherein collecting cardio-respiratory information of the subject comprises collecting cardio-respiratory information via a home sleep testing device.

4. The method of claim 1, wherein said extracting features from the cardio-respiratory information comprises extracting at least one of: heart rate variability features, respiratory variability features, or body movements.

5. The method of claim 1, wherein said collecting cardio-respiratory information of the subject comprises collecting heart rate information using a SpO2 sensor.

6. The method of claim 1, wherein said collecting cardio-respiratory information of the subject comprises collecting respiratory effort using a thoracic belt.

7. The method of claim 1, wherein said collecting cardio-respiratory information of the subject comprises collecting respiratory effort using a thoracic belt and a SpO2 sensor.

8. The method of claim 1, further comprising collecting information regarding body movement of the subject via an accelerometer.

9. The method of claim 1, further comprising determining information regarding body movement via information received from one or more of a respiratory thoracic belt and a SpO2 sensor.

10. The method of claim 1, further comprising providing an indication of one of more of the determined sleep stages to the subject.

11. A machine readable medium encoded with a computer program comprising program code for implementing the method of claim 1.

12. A computer program product including a non-transitory machine readable medium encoded with a computer program comprising program code for implementing the method of claim 1.

13. A sleep monitoring device having a processor which is programmed to carry out the method of claim 1.
